(19) United States
(12) Patent Application Publication
(10) Pub. No.: US 2010/0266694 A1
(43) Pub. Date: Oct. 21, 2010

(54) Chitosan/Carbon Nanotube Composite Scaffolds for Drug Delivery

(76) Inventors: Jessica Amber Jennings, Memphis, TN (US); Warren Oliver Haggard, Bartlett, TN (US); Joel David Bumgardner, Memphis, TN (US)

Correspondence Address: WYATT, TARRANT & COMBS, LLP, 1715 AARON BRENNER DRIVE, SUITE 800, MEMPHIS, TN, 38120-4367, US

(21) Appl. No.: 12/427,546

(22) Filed: Apr. 21, 2009

(51) Int. Cl. A61K 9/14; A61K 9/00; A61P 43/00

(52) U.S. Cl. 424/489; 424/400; 514/777; 977/742; 977/750; 977/752; 977/906

(57) ABSTRACT

A novel composite for internal application within wounds, incisions, and the like, for the prevention of biofilm growth therein is provided. Such a composite includes an antibiotic introduced within a sponge-like chitosan delivery product with electrically conductive nanomaterials present. Such a delivery product is also lyophilized subsequent to nanomaterial introduction but prior to antibiotic inclusion. The inventive lyophilized composite permits delivery of needed antibiotics internally within a patient with the simultaneous exposure to a sufficiently strong electrical current to permit a synergistic effect of increased antibiotic efficacy. In such a manner, relatively low amounts of antibiotic may be utilized to reduce the propensity of biofilm formation and/or growth within a wound or incision, or on the surface of an implant. Additionally, the lyophilized chitosan/nanomaterial composite allows for a maximum amount of antibiotic to be introduced with maximum elution therefrom as well. Lastly, the chitosan degrades over time within the subject's body, thereby avoiding any further invasive removal procedures. The method of such a manner of delivering improved antibiotic efficacy for biofilm prevention is encompassed within this invention as well.

### FIELD OF THE INVENTION

A novel composite for internal application within wounds, incisions, and the like, for the prevention and eradication of biofilm growth therein is provided. Such a composite includes an antibiotic introduced within a sponge-like chitosan delivery product with electrically conductive nanomaterials present. Such a delivery product is also lyophilized subsequent to nanomaterial introduction but prior to antibiotic inclusion. The inventive lyophilized composite permits delivery of needed antibiotics internally within a patient or externally applied to wounds with the simultaneous exposure to a sufficiently strong electrical current to permit a synergistic effect of increased antibiotic efficacy. In such a manner, relatively low amounts of antibiotic may be utilized to reduce the propensity of biofilm formation and/or growth within a wound or incision, or on the surface of an implant. Additionally, the lyophilized chitosan/nanomaterial composite allows for a maximum amount of antibiotic to be introduced with maximum elution therefrom as well. Lastly, the chitosan degrades over time within the subject's body, thereby avoiding any further invasive removal procedures. The method of such a manner of delivering improved antibiotic efficacy for biofilm prevention is encompassed within this invention as well.

### BACKGROUND OF THE INVENTION AND PRIOR ART

Infection is a frequent complication of many invasive surgical, therapeutic and diagnostic procedures, not to mention subsequent to the occurrence of various traumatic injuries. Infection often impedes the healing of wounds resulting from tissue breakdown due to extreme pressure or comorbidities such as diabetic neuropathy or venous insufficiency, causing them to become chronic. When invasive procedures are performed, whether for implantation or for actual surgical recuperative reasons, oftentimes a serious threat of further infection may exist. The source of such potential problems has become known as biofilms. These phenomena are known to occur when single-cell planktonic bacteria adhere to material or wound tissue and rapidly begin expressing extracullular polymeric substances and other proteins to form microcolonies. Within hours, microcolonies can differentiate into complex communities with defensive mechanisms against host immune cells, antibiotics, and antimicrobials. Although antibiotics can penetrate and kill sessile bacteria within a biofilm, concentrations multiple orders of magnitude higher than typical mammalian systems can withstand from a toxicity standpoint are required to eradicate such microcolony outgrowths. This makes biofilm infection extremely difficult to treat with antibiotics, primarily due to the aforementioned potential toxicity imparted by the large doses of antibiotics or antimicrobial agents that are generally required for reliable biofilm attack. Without a reliable manner of controlling, if not eliminating, such invasive biofilms, particularly without potentially further endangering the patient with the aforementioned necessarily high, possibly toxic, level of antibiotics, it may become incumbent on the medical provider to actually remove an entire implant (if one is present), or even worse, removal of a portion of a patient's body, in situations wherein the biofilm itself has taken hold and its growth cannot be suppressed without further invasive procedures. Such an instance can prove costly in terms of expense as well as quality of life, clearly.

Surfaces for bacterial and/or fungal adhesion are particularly susceptible to microbe accumulation when left within a patient's body. Damaged and necrotic tissue within wounds may become prone to biofilm initiation and growth as well. The accretion of such infectious microorganisms into biofilms creates a situation that is extremely difficult to control. Such microbes are known to attach to surfaces and secrete an enveloping polymeric matrix that ultimately protects the microorganisms from antimicrobial attack and immune cells. The subject biofilm then can be protected sufficiently to the extent that individual film components can multiply and disperse for transport throughout the patient's body (through what is termed planktonic bacteria), leading to infections in different areas, but due, ostensibly, to the initial biofilm production itself (the biofilm is termed sessile bacteria). These other infections become the target of treatment thereafter, but the source of their growth, the biofilm, may remain viable. As such, the necessity of biofilm eradication is of utmost importance, rather than the subsequent treatment of further infections. Without prevention or control of the biofilm, further infections will continue to arise, in essence.

As noted above, sessile bacteria (biofilms) develop and grow most readily on inert surfaces or on non-living tissue (such as medical implants and/or dead tissues). It appears that most invasive infections within a patient's body are due to sessile bacteria growth. In such instances, the biofilm, being in a protective environment from immune system attack, will generate planktonic bacteria that will freely transfer throughout a patient's body and settle in suitable locations for further growth and reproduction. Without a proper manner of preventing planktonic bacteria development and movement, particularly from a sessile bacteria source, the chances of widespread infection are relatively high. The pattern of biofilm development involves initial attachment of a microorganism to a solid surface, the formation of microcolonies attached to the surface, and finally the differentiation of the microcolonies into exopolysaccharide-encased mature biofilms. The aforementioned planktonic bacterial cells are released from biofilms in a natural pattern of programmed detachment, so that the biofilm serves as the source for multiple, recurrent acute invasive infections. Antibiotics may viably treat the infection caused by the planktonic bacteria, but fail to kill the sessile bacteria itself; thus, rendering a potential cycle of invasive infection without proper treatment and destruction of the biofilm bacteria source.

Such sessile bacteria are also known to release antigens and stimulating antibody production that activates a patient's immune system to attack the biofilm and its surrounding tissues. As the biofilm is within a protective environment, this signal may deleteriously create a situation wherein the generated antibodies and the cytotoxic byproducts thereof attack the patient's cells and tissues, particularly since the biofilm is protected from such immune system responses. Clearly, in such a situation, prolonged inflammation creates even more problems for the patient that require further medical attention.

As it is, the generation and growth of sessile bacteria in biofilm formation, and the subsequent proliferation possibly of planktonic bacteria therefrom, is highly problematic in the medical community. The defensive measure of utilizing synthetic antibiotics to prevent biofilm formation, or at least to attack and destroy a biofilm itself after initial generation, would be highly effective but for the potential dangers with introducing the necessarily large amounts of antibiotics for such a procedure to function properly. Such large dosages of antimicrobial materials leads to, as noted above, the further problem of toxic levels accumulating in a patient's body and bloodstream. Toxic levels of antibiotics can have severe side effects such as hearing loss or kidney failure, as examples. Hence, there exists a need to provide an effective manner of antibiotic delivery locally to the site of injury for biofilm prevention and/or elimination, while limiting the amount of systemic antibiotic to acceptably non-toxic levels, but in an amount that will actually function as needed to destroy sessile bacteria. Unfortunately, no such delivery device or method has been provided the medical community that permits such a targeted antibiotic level for safe treatment in that manner. Furthermore, the ability to provide a one-time therapeutic invasive antibiotic treatment, without the need for further removal thereof, would be of great interest to the medical community. To date, again, no such beneficial product or method has been made available.

### SUMMARY AND ADVANTAGES OF THE INVENTION

Therefore, it is an advantage of this invention to provide a safe and reliable antibiotic delivery system for individual patients. Another advantage of the invention is the ability to provide a one-time invasive procedure for introduction of a composite therapeutic agent delivery system wherein the therapeutic agent elutes therefrom steadily and continuously until depleted. A further advantage of this invention is the ability to provide a safe, non-cytotoxic delivery device that permits an increase in antibiotic efficacy internally during utilization.

Accordingly, this invention encompasses a therapeutic agent delivery composite comprising a lyophilized chitosan/electrically conductive nanomaterial (preferably carbon nanotubes) sponge-like combination and at least one liquid therapeutic agent (such as an antibiotic, including an antifungal and/or antiviral agent) present therein. Thus, also encompassed within this invention is a method of producing a sponge-like liquid therapeutic agent delivery device, said method comprising the steps of providing powdered chitosan; b) providing electrically conductive nanomaterials; c) mixing said chitosan and said nanomaterials together in the presence of a weak acid to form a gel mixture; d) freezing the resultant mixture; and e) lyophilizing the frozen mixture of step “d” to form a sponge-like composite of chitosan and electrically conductive nanomaterials.

The chitosan component exhibits a certain degree of antibacterial efficacy itself, but is present primarily for the purpose of delivering the therapeutic agent, such as, as one non-limiting example, an antibiotic (in liquid state), into or on the surface of the subject wound or incision. Additionally, chitosan degrades over time and, being non-toxic to mammals, is degraded thereafter within the tissue. As such, the utilization of such a carrier base avoids any further invasive procedures to remove the delivery device from the subject patient.

The electrically conductive nanomaterial is necessary to permit an electrical current to be supplied to the overall composite after introduction (implantation) within the subject patient. The nanomaterial is arranged and configured in such a manner to provide proper percolation capability for an externally or internally generated electrical current to be applied without excessive heat generation. In such a manner, it has been found that the efficacy of the antibiotic component itself is increased to levels well above that for the antibiotic present alone. Thus, through this electrical current exposure the amount of antibiotic present within the overall delivery composite may be reduced to safe, non-toxic levels, and yet still be strong enough (through the synergistic application of the electrical current therethrough) to properly attack and destroy (or inhibit the growth of) sessile bacteria in a biofilm.

The overall composite must be in a lyophilized (freeze-dried) form in order to provide an effective reservoir for liquid antibiotic delivery. As well, in order to properly provide the static network of nanomaterials within the composite for proper electrical conductivity to exist, such nanomaterials must also be present during such a lyophilization step. If added subsequent to lyophilization, such nanomaterials would require adhesion to the lyophilized sponge-like chitosan and proper arrangement in network format as well. Such a manner of introducing such nanomaterials would be extremely difficult, time-consuming, and expensive to accomplish, if at all possible.

The resultant sponge-like composite thus can hold the needed amounts of antibiotic (or other therapeutic agent) safely and securely for introduction within a wound or incision. Subsequent to such introduction, the sponge-like quality of the composite facilitates even elution of the entire liquid antibiotic supply at a controlled, continuous rate. Additionally, the lyophilized composite allows for conductance of the electrical current in order to expose the underlying or surrounding tissue thereto for the desired efficacy increase. In such a manner, with the electrical current application provided to the securely, but steadily eluted, antibiotic, the desired level of increased antibiotic efficacy may be achieved while ensuring even application of the antibiotic itself to the target biofilm area.

### DETAILED DESCRIPTION OF THE INVENTION

For the purposes of this invention, the term “sponge-like” is intended to mean a solid form that exhibits properly sized pores that retain liquid antibiotic materials in place through adhesion and cohesion, as well as exhibits low degrees of hysteresis upon application of force thereto. Preferably, the pore sizes are targeted for such a sponge-like composite to be from 10 to 1000 microns; the introduction of electrically conductive nanomaterials prior to lyophilization appears to actually reduce the pore size, thereby allowing for potentially greater reliability in holding capacity of liquid therapeutic agents prior to and during delivery. As well, the overall dimensions of such a composite may be of any measurements to permit introduction within any desired size wound or incision as long as the liquid antibiotic is retained as described above and eluted as well as desired and the electrically conductive nanomaterials are properly aligned therein to permit sufficient current to be applied thereto in contact with the liquid antibiotics themselves. As noted above, “lyophilized” is intended to mean freeze-dried. The actual lyophilization procedure is described in greater detail in the examples below. The term “electrically conductive nanomaterials” is intended to mean a material of sub-micron dimensions that exhibits electrical conduction through a suitably connected network. The term “antibiotic (or drug) delivery system” is intended to encompass a structure including components for complete delivery of antibiotics (or other pharmaceuticals) from the composite structure and into a user's body.

As noted above, the potential for antibiotic toxicity when present in relatively large amounts within a patient has prevented the introduction of large amounts of such materials for biofilm reduction/control. Without a manner of providing suitable biofilm attack while ensuring undesirable toxicity problems internally for a target patient, proper biofilm antibiotic-based therapeutic methods have been either avoided or ineffective.

A phenomenon has recently been discovered wherein the in vitro application of electrical currents to biofilms may increase the efficacy of antibiotics against biofilms. Exposure to electrical currents without antibiotics present has also been shown to disrupt biofilms and kill sessile bacteria, however this requires prolonged exposure times and high intensity levels to achieve the same effects as when antibiotics or antimicrobials are included. In such a proposed procedure, differing current waveforms have been applied to biofilms in the presence of antimicrobial/antibiotic compounds in very low voltages thereby reducing the concentration necessary of such compounds to impart highly effective bacteria kill levels. In essence, it has been theorized that low concentrations of antibiotic compounds may be employed with an electrical current applied thereto to generate a bacteria kill rate equal to a usual concentration of antibiotic an order of magnitude greater.

Although such a phenomenon has shown great promise, again, theoretically, the actual potential for implementation of such an electrical current has been very difficult to accomplish. Initially, a controlled low voltage current application on a continuous basis to tissue through a material present to protect tissue, serve as scaffolding for tissue ingrowth, or deliver therapeutic agents is not a simple task. The retention of an antibiotic for subsequent delivery into targeted cells and/or tissues must be undertaken characteristic of the material, as well as the ability to apply the desired level of electric field to the biofilm infected tissue without necrotizing surrounding tissues or using excessive current densities. Furthermore, the application of an electrical field alone to targeted biofilm bacteria has proven ineffective, as has the treatment of such bacteria with low concentrations of antibiotics, etc. Thus, the coupling of both the low antibiotic concentration and an electrical current is necessary to achieve the synergistic effects to impart the desired biofilm prevention levels.

Additionally, it was realized that further trauma to a patient would occur if a delivery device for such a combination of electrical current and antibiotic were implanted within a patient needing future removal. Thus, with all of these variables at issue, it was difficult to determine the best approach to overcome all of these potential problems. With the seriousness of biofilm generation and its subsequent hand in causing widespread infections within patients, however, the ability to provide an effective sessile bacteria control/elimination method and device was imperative.

The resulting development was, as noted above, a lyophilized chitosan/electrically conductive nanomaterial composite with liquid antibiotic present therein. Such an inventive composite permits the concurrent delivery of antibiotic into a wound or incision with an electrical current, conducted through the chitosan base via a network of such nanomaterials configured to provide the necessary percolation of current through the chitosan and into the tissue and biofilm present (of course, any antibiotic in contact with the nanomaterial network would be exposed to the electrical current as well). The sponge-like composite of chitosan and nanomaterials provides a porous structure in which the liquid antibiotic (a term intended to include antimicrobial, antifungal, and antiviral agents as well) is securely held for transfer with the composite into a wound or incision and can easily elute therefrom after implantation. Furthermore, the chitosan base exhibits the ability to easily degrade after time and thus clears from the patient's body without any toxic or otherwise debilitating or undesirable effects.

Chitosan (and chitosan-metal compounds) are known to provide antimicrobial activity as bactericides and fungicides, as well as antiviral activity. Chitosan is the commonly used name for poly-[1-4]-β-D-glucosamine. Such a compound is chemically derived from chitin (a poly-[1-4]-β-N-acetyl-D-glucosamine) which, in turn, is derived from the cell walls of fungi, the shells of insects, and, especially, crustaceans (i.e., it is widely available and is generally inexpensive to manufacture). Chitosan materials have been known to be treated with a solution of zinc sulfate, cupric sulfate, or silver nitrate to enhance antimicrobial activity. In this invention, the chitosan component does not require any such treatment for utilization; however, if desired, and if such treatment withstands lyophilization to form a suitable sponge-like form, then such treatment may be employed.

The electrically conductive nanomaterials are, as the name implies, intended to be materials that are sub-micron in size (preferably with long lengths and small diameters) that exhibit electrical conductance. Although any number of materials may meet such a description, the preferred materials are carbon nanotubes. Such materials are produced through a number of possible methods, although arc discharge and vapor deposition are considered the most readily available manufacturing processes. Such nanotubes appear to have hexagonal base units in sheet form; the sheets are either single layer (single-walled) and form actual tubes when one end of such a sheet is reacted and chemically bonded to the other end. Other nanotubes are formed through the creation of a sheet of repeating hexagonal units of carbon (much like fullerenes, but in flat, instead of spherical form) with the sheet then rolled up into a tube of multiple layers (multi-walled nanotubes). Again, the diameter of such tubes should be very small while the length should be very long. The arrangement of multiple nanotubes within and throughout the target chitosan base allows for an electrical current to be applied and transferred through the chitosan itself. The difficult part has been the actual attainment of a sufficient network for such a conductive system to exist, while also ensuring that introduction within the chitosan base properly occurs. For instance, single-walled carbon nanotubes are excellent (and superior) electrical conductors, but are very difficult to disperse in liquid to accord a uniform network.

To do so, it was realized that such nanotubes required functionalization on their surfaces to improve their liquid solubility. For proper control and dispersion, some type of mechanism was proven necessary to provide the necessary network; the utilization of nanosized materials is extremely difficult, particularly when controlled arrangements and configurations are the overall aim within a porous matrix. The distance across the overall chitosan base over which the network of nanosized materials must percolate is, in comparison with the sizes of the individual nanomaterials themselves, vast. Thus, as alluded to above, the proper control to effectuate the necessary percolation network throughout the chitosan base required proper surface treatments, as well as sufficient amounts of nanomaterials themselves, to best ensure proper network construction was obtained. Thus, strong acid treatments (such as concentrated hydrochloric acid, nitric acid, and/or sulfuric acid) and sonication were applied to purify the nanomaterial surfaces. Depending on the types of acids and solvents used, functional groups such as carboxylic, amide, or hydroxyl groups are introduced onto the ends of nanotubes and at defect sites on the side walls. While these harsh treatments can damage the nanotubes and effectively decrease electrical conductivity, they afford greater increases in the liquid solubility of the resultant treated nanomaterials. In such a manner, nanotubes were functionalized and washed and filtered with distilled water to purify and neutralize pH. Afterward, nanotubes were dried in a high temperature oven to remove water content and facilitate accurate weighing. Functionalized nanotubes were then dispersed in an aqueous weak acetic acid solution and mixed with chitosan powder (prior to and/or during freeze-drying) in order to effectuate the desired network formation. Alternatively, it has been found that sonication of the nanotubes prior to and/or during chitosan base introduction permitted formation of a network suitable for electrical current application as well. In general, the carbon nanotubes (electrically conductive nanomaterials) are introduced within the pre-lyophilized chitosan base in a concentration of from about 0.05 to about 5 mg/ml (aqueous solution); preferably, the range is from 0.25 to 2 mg/ml. Single-walled nanotubes are preferred due to the superior electrical conductance properties exhibited by them, although some multi-walled tubes exhibit electrical conductivity as well. Additionally, as noted above, the longer and thinner the nanotubes, the lower concentration is needed to reliably form a conductive interconnected network such can form (ostensibly due to their long lengths and the propensity to contact other nanotubes when in dispersion as a result of such long lengths, thereby providing the necessary overlap for proper percolation to occur).

The chitosan/nanomaterial composite was then lyophilized to reduce the rigidity of the chitosan base and allow for the nanotubes to become enmeshed throughout the entire composite structure in network form. Such lyophilization may be performed in any acceptable freeze-drier, such as a LABCONCO® freeze-drier, at, as one potentially preferred, non-limiting, level, 0.06 mbar for 48 hours. The resultant composite exhibited a porous structure and a spongy state, as well as a dark color (such as grey or even black, depending on the amount of nanotubes present; being preferably carbon in nature, the color of the nanotubes dispersed throughout the target composite structure dominated the typical whitish/yellowish color of the chitosan itself). The lyophilized composite also exhibited an electrical conductivity measurement in excess of that for the chitosan alone (as described below). In such form, the introduction of a liquid would allow for large amounts thereof to be securely held within the pores of the sponge-like composite, as well as the ability to maneuver such a composite within a wound or incision as the composite itself was sufficiently flexible to become introduced within any type of cavity. The loose porous structure then permits elution therefrom continuously, but at a controlled rate, subsequent to liquid introduction and implantation within or external placement over a wound or incision. Upon such implantation, the wound or incision may be sealed with a suture, staple, or other type of procedure, and the wound may be externally dressed to prevent surface infection or entry of bacteria from outside the patient's body through the resultant open skin. As noted, optionally, the antibiotic-containing composite may be applied itself to an external wound or incision instead.

The antibiotic components should be one or more that is water soluble in order to provide a liquid thereof to facilitate introduction within and thus delivery from the lyophilized sponge-like chitosan/electrically conductive nanomaterials composite. Thus, any such water-soluble drug may be utilized for such a purpose, preferably of a type that does not exhibit to strong a charge (if above a certain level, the antibiotic may lose its viability when exposed to the electrical current, in essence, or such a drug may elute from the construct at increased rates through electrophoretic mechanisms). Suitable drugs for delivery therewith the inventive composite include, without limitation, those belonging to the following groups: aminoglycosides (Kanamycin, Neomycin, and the like), Rifampin, cephalosporins and related beta lactams (Cefazolin, Cefuroxime, Cefaclor and the like), glycopeptides (Amikacin, Vancomycin and the like), penicillins (amoxicillin, ampicillin, carbenecillin, cloxacillin, dicloxacillin, and the like), quinolones (gatifloxcin, ciprofloxacin and the like), sulfonamides (sulfadiazine, sulfamethoxazole, sulfamerazine, trimethoprim, sulfanilamide, and the like), tranquilizers such as, e.g., diazepam, droperiodol, fluspirilene, haloperidol, lorazepam, and the like; antiviral agents such as, e.g., idoxuridine, acyclovir, vidarabin, and the like; antibiotic agents such as, e.g., clindamycin, erythromycin, fusidic acid, gentamicin, and the like; antifungal agents such as, e.g., miconazole, ketoconazole, clotrimazole, amphotericin B, nystatin, and the like; and antimicrobial agents such as, e.g., metronidazole, tetracyclines, and the like.

The active substances mentioned above are also listed for illustrative purposes; the invention is applicable to any pharmaceutical formulation regardless of the active substance or substances incorporated therein. Although the desired function is for biofilm reduction, the inclusion of any such water-soluble therapeutic agent (such as those listed above in terms of antibiotic, antimicrobial, antiviral, and/or antifungal types) is suitable for other medicinal purposes as well. It is believed, without any reliance upon a specific scientific theory, that the antibiotic, antimicrobial, and the like, components noted above exhibit the desired increased efficacy upon exposure to an applied electrical current via the nanomaterial conductive network within the composite due to the effect of the electrical current on the surrounding tissues and/or cells to be treated. Thus, in terms of the amount of therapeutic agent(s) present, the exposure of the target wound and/or incision area to such electrical currents permits lower amounts of such therapeutic agents to be present for effective therapeutic benefit to take place.

The concentration of the therapeutic (antibiotic, for example, again) component (the dose) within the delivery composite will depend primarily upon the desired degree of treatment sought for the target user. Furthermore, the antibiotic concentration should be at a level that is considered non-toxic (in case of excess antibiotic eluting from the composite and into the patient's body), but sufficient, will apply electrical current thereto, to provide the desired level of antibiotic efficacy for biofilm attack. In general, the concentration of antibiotic in an aqueous solution would be from about 0.1 to about 10 mg/ml concentrated solution. In terms of adding such an aqueous solution to the lyophilized chitosan/nanomaterial composite, such would be from about 0.01 to about 10% by weight of the composite; from 0.1 to about 5% is potentially, though not necessarily, preferred.

The application of an electrical current then can be undertaken after the entire antibiotic (etc.)-containing composite is implanted within a wound or incision and such may then be sealed. Either an external or internal electrical source may be utilized, with a very weak current applied. The current source may take the form of a battery powered power source with leads and electrodes applied to the construct and also to the surrounding tissue. Devices generating wireless radio-frequency fields or pulsed electromagnetic fields may also be used to provide the electrical stimulation component of this therapy. The direct current field strength should be from about 50 to about 300 mV/mm, preferably from about 100 to about 200 mV/mm, and most preferably about 150 mV/mm, and the field density should be from about 75 to about 125 microAmps/cm² (preferably, about 100 microAmps/cm²).

If desired, the antibiotic delivery composites may include other types of materials and/or compounds for introduction within the target wound and/or incision, including analgesics, growth factors, antioxidants, or living cells, as examples.

### PREFERRED EMBODIMENTS OF THE INVENTION

The invention is hereinafter more particularly described through the following non-limiting examples. It is noted that specific antibiotics are utilized within these examples; however, it should be well understood by the ordinarily skilled artisan within the pertinent art that the inventive method may be practiced with any known water-soluble antibiotic. Thus, the specific types listed below are in no way intended to indicate a limitation as to the breadth of this invention.

### Lyophilized Chitosan/Carbon Nanotube Composite Production

The inventive materials were formed by adding nanotubes (in powder form) to an acid solvent (such as, without limitation, acetic acid, nitric acid, sulfuric acid, oxalic acid, hydrochloric acid, and the like, and mixture thereof) to which chitosan powder is added. The concentration and morphology of nanotubes within the composite was controlled, as noted above, by functionalization with various functional groups prior to mixing and sonication. Mixtures of chitosan and nanotubes in weak acids (acetic acid, as one non-limiting example) formed a gel mixture with viscosity that varied according to chitosan weight percent. Characteristics of this gel and constructs made from this material such as crystallinity, degradation, and mechanical properties were dependent on the degree of deacetylation (DDA) of chitosan as well as the type of acid used during dissolution. To form the sponge-like construct from this material, the gel was frozen in blocks and then lyophilized (freeze-dried). Material properties were controllable by altering freezing temperature, freezing rate, and lyophilization technique. After lyophilization, the sponge-like material was (preferably, though not necessarily) neutralized in a suitable basic solution to prevent dissolution thereafter. As noted above, the finished composite exhibited the color dominated by that of the nanomaterials (dark grey, black, etc.) as well as the electrical conductivity of the nanomaterials embedded within the lyophilized chitosan composite, rather than the chitosan alone.

### Example 1

### Nanotube Functionalization

One gram of single-walled carbon nanotubes (Sigma) in powder form were mixed into a mixture of one liter of a 3:1 mixture of sulfuric acid:nitric acid (full strength). In a chemical fume hood, these components were placed in a sonicating water bath with heat application for one hour. Flasks were then set aside in the fume hood overnight, during which time solid nanotubes settled to the bottom. Excess acid without nanotubes was then decanted and the mixture was diluted with distilled water. This mixture was then filtered through a polycarbonate filter with a 0.2 micron pore size to recover nanotubes from the acid solution. The nanotubes were washed and resuspended in water and filtered again. This process was repeated until the wash solution reached neutral pH. To remove water content from nanotubes, which facilitates weighing, the filtered solid was transferred to an aluminum dish, covered with a ventilating top and placed in a heated oven overnight.

Alternatives to this functionalization method are also possible, including fluorination or the use of less concentrated acids combined with refluxing, among others.

### Examples 2-3

### Composite Fabrication

Fifty mg of nanotubes were added to 50 mL of 1% v/v acetic acid. This mixture was sonicated in a heated water bath for 3 hours to improve dispersion (degree of dispersion was estimated by observation of the liquid mixture for color, opacity, and presence of aggregated material). To this solution was added 1.5 g chitosan with 61% degree of deacetylation to make a 3% by weight mixture. This mixture was placed in a non-heated water bath and sonicated for one hour to both encourage dispersion of nanotubes and to facilitate the dissolution and degassing of chitosan mixtures.

Using a 25 mL pipet and pipet-aid 20 mL of mixtures were withdrawn and placed into aluminum weigh dishes 6 mm in diameter. Dishes were placed on a level surface in a −80° C. freezer overnight. The freeze-drier was activated on and allowed to reach freezing temperatures before samples were placed in the chamber on a level surface. Vacuum was activated immediately to minimize sample melting, and continued building vacuum until 0.06 mbar was reached. Samples were lyophilized at these parameters for 48 hours and then removed from the chamber.

To neutralize constructs, they were removed from aluminum dishes and immersed in 2M sodium hydroxide solution for 5 minutes with stirring. Constructs were washed with water and rinsed repeatedly until neutral pH of wash solution was achieved. To maintain porous structure, wet sponge-like constructs were placed in a −80° C. freezer overnight and relyophilized for 24 hours at the above mentioned parameters.

Example 2 included single-walled nanotubes; Example 3 included multi-walled nanotubes. Both composite examples exhibited greyish color.

### Antibiotic Composite Introduction

In one example three different types of constructs were fabricated in the manner described above all with 3 wt % chitosan: 1 mg/ml single-walled functionalized tubes (Example 2, above)(SWNTs), 1 mg/ml multi-walled functionalized tubes (Example 3, above)(MWNTs), and no nanotubes (a Comparative Example). Each sponge-like block was cut into approximately equal pieces and individually weighed. No statistical differences in average weight were found (Table 1).

TABLE 1

| Construct type | average weight (g) | standard deviation |
|---|---|---|
| SWNTs | 63.6 | 3.5 |
| MWNTs | 61.5 | 6.6 |
| plain chitosan | 63.8 | 3.5 |

Two different antibiotic solutions, vancomycin and amikacin (different classes of antibiotics), were reconstituted from powder in phosphate buffered saline (PBS) at a concentration of 5 mg/mL. Five mL of these solutions were placed in different small plastic weigh boats, and constructs were placed in these solutions for 5 minutes to absorb liquid contents into the porous structure. The amount of fluid remaining after removal of the construct was measured and used to determine uptake. No detectable differences in uptake were found between nanotube-containing scaffolds and plain scaffolds, as shown in Table 2.

TABLE 2
Liquid Therapeutic Agent Uptake in
Lyophilized Chitosan/Nanomaterial Composites

| | | | vancomycin | |
|---|---|---|---|---|
| | amikacin | | average | |
| | average uptake | | uptake | |
| | ml/g | st. dev | (ml/g) | st. dev |
| SWNTs | 24.40765 | 1.612348 | 22.77932 | 1.13813 |
| MWNTs | 23.96494 | 1.888182 | 25.28903 | 4.000468 |
| plain | 23.95768 | 1.838647 | 22.23764 | 1.967187 |
| chitosan | | | | |

### Electrical Current Application

Electrical currents have been applied to constructs in hydrated and non-hydrated forms to measure conductivity. Electrical measurements have been made using the four-point probe technique, in which a constant current is applied between two electrodes and voltage drop between inner electrodes is used to calculate conductivity. Hydrated constructs demonstrate much greater conductivity than dry constructs, because charges are ionically transferred. Constructs were hydrated with saline at physiological levels to replicate clinical conditions.

In the following example, commercially available multi-walled nanotubes (MWNT) from Sigma were evaluated for potential use in chitosan composites. These tubes were assigned identification letters for identification and had the following properties for length, outer diameter (OD) and inner diameter (ID).
A. OD=20-30 nm; ID=5-10 nm; length 0.5-200 um (thin, long)
B. OD=10-30 nm; ID 3-10 nm; length 1-10 um (thin, short)
C. Diam=110-170 nm; length 5-9 um (thick, short)
   The composites were made in the same manner as for Example 3, above, but at the different concentrations listed below in Table 2 (some had 0 concentration, thereby pertaining to chitosan-only lyophilized sponge-like composites. Conductivity of constructs made with varying concentrations of these types of tubes is shown in Table 3, wherein the composites made from such carbon nanotubes were measured by a four point probe technique (using four equally spaced electrodes, a constant current is applied to the outer two electrodes and resultant voltage drop is measured for the inner two). These results demonstrate that not all nanotubes are suitable for inclusion in these constructs in order to improve electrical properties. The higher the measured S/cm³ the more suitable a conductor the composite was measured to be the lower the resistivity, the higher the conductivity). A custom-made probe with four equally spaced silver electrodes was inserted into each of the composites. A constant current was applied to the two outer electrodes and the voltage between the inner electrodes was used to calculate resistivity (inverse of conductivity) by the equation resistivity=2*π*distance between electrodes* (voltage drop/applied current). Conductivity measurements may also be made using impedance analysis techniques.

TABLE 3
Composite Electrical Conductivity With and
Without Nanotubes

| | concentration | conductivity |
|---|---|---|
| | nanotubes | (S/cm) |
| nanotube | mg/ml in | across |
| type | composite | composite |
| “A” | 0 | 2.5E−02 |
| tubes | 0.5 | 1.7E−02 |
| | 1 | 7.2E−03 |
| | 1.5 | 7.2E−03 |
| “B” | 0 | 2.5E−02 |
| tubes | 0.125 | 1.8E−02 |
| | 0.25 | 2.3E−02 |
| | 0.5 | 2.2E−02 |
| | 1 | 2.9E−02 |
| “C” | 0 | 3.0E−02 |
| tubes | 0.4 | 2.2E−02 |
| | 0.5 | 2.5E−02 |
| | 0.6 | 6.2E−02 |
| | 0.8 | 6.9E−02 |
| | 1 | 8.2E−02 |

Thus, the A tubes and B tubes did not work well in terms of conductivity within the chitosan composite; however, the C tubes exhibited excellent conductivity results, thus providing a suitable source for such a property within the target composite. (Both multi-walled and single-walled types meeting these dimensions meet the necessary conductivity requirements.) The electrically conductive nanomaterials must impart an electrical conductivity to the overall lyophilized chitosan/nanomaterials composite at least 1.5 times greater than a lyophilized chitosan composite alone at a nanomaterials concentration of at least 0.6 mg/ml within the composite. Thus, the inventive composites including such types of nanomaterials exhibited far improved electrical conductance measurements than for lyophilized chitosan composites without nanotubes present.

### Bacteria Reduction Measurements

Composites were then prepared through hydration in amikacin or vancomycin to introduce the liquid therapeutic agents (here antibiotics) into the sponge-like composites. Composites from Table 2 were placed in glass Wheaton vials in 10 mL of PBS and were incubated at 37° C. At 4, 24, 48 and 72 hours, 2 mL of eluate was removed and stored at −20° C. Antibiotic concentration in these eluates were measured using immunofluorescence (TDx Abbott). Elution profiles for each composite scaffold were determined and the measured results shown in Table 3.

TABLE 3
Elution of Certain Antibiotics from Composites

concentration μg/ml

| | | | |
|---|---|---|---|
| Hours | type | average | stdev |

Vancomycin elution

| | | | |
|---|---|---|---|
| 4 | SWNTs | 413.4 | 96.35217 |
| | MWNTs | 474.75 | 107.3652 |
| | plain | 377.925 | 80.57718 |
| | chitosan | | |
| 24 | SWNTs | 197.075 | 53.80622 |
| | MWNTs | 215.175 | 34.35961 |
| | plain | 253.25 | 83.94556 |
| | chitosan | | |
| 48 | SWNTs | 35.705 | 3.475644 |
| | MWNTs | 32.2125 | 7.021758 |
| | plain | 36.2175 | 12.56331 |
| | chitosan | | |
| 72 | SWNTs | 8.6 | 1.124189 |
| | MWNTs | 5.65 | 1.415721 |
| | plain | 6.7025 | 1.762751 |
| | chitosan | | |

Amikacin elution

| | | | |
|---|---|---|---|
| 4 | SWNTs | 555.375 | 48.99309 |
| | MWNTs | 668.175 | 41.02758 |
| | plain | 605.475 | 69.1899 |
| | chitosan | | |
| 24 | SWNTs | 214.925 | 14.01746 |
| | MWNTs | 180.8 | 24.92241 |
| | plain | 180.975 | 54.73727 |
| | chitosan | | |
| 48 | SWNTs | 27.79 | 2.04413 |
| | MWNTs | 23.365 | 6.434887 |
| | plain | 20.685 | 6.478217 |
| | chitosan | | |
| 72 | SWNTs | 7.01 | 0.41497 |
| | MWNTs | 4.4375 | 1.014573 |
| | plain | 3.77 | 1.334141 |
| | chitosan | | |

The results show compatibility in terms of available antibiotic eluted from the composite, particularly in comparison with chitosan composites with no nanomaterials present.

Eluates taken from constructs of these different types were tested for bactericidal activity in turbidity tests against appropriate strains of bacteria: amikacin eluates against Pseudomonas aeruginosa and vancomycin eluates against Staphylococcus aureus. Eluates were added to trypticase soy broth in a 1:10 dilution. Broth was then inoculated with bacteria and then incubated overnight at 37° C. Absorbance of these solutions (turbidity) was used as an indicator of bacterial growth. Percent inhibition of bacterial growth is calculated from these measurements. The results are provided in Table 4 and demonstrate that presence of nanotubes does not inactivate antibiotics, and that elution of amikacin is inhibitory up to 24 hours and vancomycin up to 48 hours.

TABLE 4
Antibiotic Eluate Activity
Vancomycin eluate
activity

% inhibition of
S. aureus

| Hours | type | average | stdev |
|---|---|---|---|
| 4 | SWNTs | 98.15395 | 0.87008 |
| | MWNTs | 98.67642 | 0.159616 |
| | plain | 98.25845 | 0.493816 |
| | chitosan | | |
| 24 | SWNTs | 98.9899 | 0.159616 |
| | MWNTs | 98.67642 | 0.525938 |
| | plain | 98.88541 | 0.594174 |
| | chitosan | | |
| 48 | SWNTs | 98.46743 | 0.159616 |
| | MWNTs | 98.74608 | 0.552926 |
| | plain | 98.11912 | 0.455475 |
| | chitosan | | |
| 72 | SWNTs | 68.05991 | 53.51209 |
| | MWNTs | 2.890979 | 17.68911 |
| | plain | 66.7015 | 55.77435 |
| | chitosan | | |

Amikacin eluate
activity

% inhibition of
P. aeruginosa

| Hours | type | average | stdev |
|---|---|---|---|
| 4 | SWA | 104.0667 | 0.186359 |
| | CA | 104.3514 | 0.49306 |
| | PA | 104.9207 | 0.281749 |
| 24 | SWA | 103.782 | 1.406981 |
| | CA | 106.1 | 3.07916 |
| | PA | 104.5547 | 0.140874 |
| 48 | SWA | 89.95527 | 3.80817 |
| | CA | 95.93331 | 3.860574 |
| | PA | 63.94877 | 33.88804 |
| 72 | SWA | 7.889423 | 0.531789 |
| | CA | 13.42012 | 13.87827 |
| | PA | −0.12196 | 9.061243 |

Thus, such therapeutic agents are not only compatible with the inventive chitosan/nanomaterial composites, but such delivery devices properly elute sufficient amounts of antibiotics to sufficiently kill target microbes as desired, thus indicating the availability and viability of such composites as drug delivery devices for wounds and/or incisions. Upon connection to a suitable electrical source, subsequent to wound or incision placement (internally or externally), such a device should provide effective therapeutic benefits, particularly biofilm reduction and/or prevention.

While certain preferred and alternative embodiments of the invention have been set forth for purposes of disclosing the invention, modifications to the disclosed embodiments may occur to those who are skilled in the art. Accordingly, this specification is intended to cover all embodiments of the invention and modifications thereof which do not depart from the spirit and scope of the invention.

What is claimed is:

1. A therapeutic agent delivery composite comprising a lyophilized chitosan/electrically conductive nanomaterial sponge-like combination and at least one liquid therapeutic agent present therein.

2. The composite of claim 1 wherein said electrically conductive nanomaterial comprises carbon nanotubes.

3. The composite of claim 2 wherein said carbon nanotubes are multi-walled or single-walled in structure.

4. The composite of claim 1 wherein said therapeutic agent is selected from the group consisting of at least one antibiotic, at least one antimicrobial, at least one antifungal, at least one antiviral, and any combinations thereof.

5. The composite of claim 4 wherein said therapeutic agent is at least one antibiotic.

6. The composite of claim 4 wherein said electrically conductive nanomaterial comprises carbon nanotubes.

7. The composite of claim 5 wherein said electrically conductive nanomaterials comprises carbon nanotubes.

8. The composite of claim 6 wherein said carbon nanotubes are single-walled or multi-walled in structure.

9. The composite of claim 7 wherein said carbon nanotubes are single-walled or multi-walled in structure.

10. A method of producing a sponge-like liquid therapeutic agent delivery device, said method comprising the steps of:
a) providing powdered chitosan;
b) providing electrically conductive nanomaterials;
c) mixing said chitosan and said nanomaterials together in the presence of a weak acid to form a gel mixture;
d) freezing the resultant mixture; and
e) lyophilizing the frozen mixture of step “d” to form a sponge-like composite of chitosan and electrically conductive nanomaterials.

11. The method of claim 10 further comprising the following sequential steps:
f) providing a sample of liquid therapeutic agent; and
g) dipping said sponge-like composite into said liquid therapeutic agent for a sufficient time to allow for uptake of at least some of said liquid therapeutic agent into said sponge-like composite.

12. The method of claim 10 wherein the electrically conductive nanomaterials of said sponge-like composite include carbon nanotubes.

13. The method of claim 12 wherein said carbon nanotubes are multi-walled or single-walled in structure.

14. The method of claim 11 wherein the electrically conductive nanomaterials of said sponge-like composite include carbon nanotubes.

15. The method of claim 12 wherein said carbon nanotubes are multi-walled or single-walled in structure.

16. The method of claim 11 wherein said therapeutic agent is selected from the group consisting of at least one antibiotic, at least one antimicrobial, at least one antifungal, at least one antiviral, and any combination thereof.

17. The method of claim 16 wherein said therapeutic agent is at least one antibiotic.
